(19) United States
(12) Patent Application Publication
(10) Pub. No.: US 2022/0143291 A1
(43) Pub. Date: May 12, 2022

(54) WHOLE BLOOD TREATMENT DEVICE AND METHODS OF REMOVING TARGET AGENTS FROM WHOLE BLOOD

(71) Applicant: Qualigen Inc., Carlsbad, CA (US)

(72) Inventor: Michael Poirier, Vista, CA (US)

(21) Appl. No.: 17/434,107

(22) PCT Filed: Feb. 26, 2020
(86) PCT No.: PCT/US2020/019993

§ 371 Date: Aug. 26, 2021

Related U.S. Application Data
(60) Provisional application No. 62/810,870, filed on Feb. 26, 2019.

(51) Int. Cl. A61M 1/36; A61P 39/06

(52) U.S. Cl. CPC A61M 1/3679; A61P 39/06

(57) ABSTRACT

A whole blood treatment device includes a cartridge configured to receive whole blood, having a wall defining an interior volume, an inlet, and an outlet, a support structure having a surface, inside of the cartridge, and an affinity agent, attached to the surface of the support structure. The affinity agent is effective to bind to a target agent that is desirable for removal from a patient. The target agent is selected from the group consisting of: inhibitory checkpoint molecules, inflammatory factors, cancerous cells, autoantibodies, opioids and heavy metals. A method of removing a target agent from whole blood of a patient in a whole blood treatment device comprising pumping whole blood into a cartridge, containing a support structure having a surface, with a plurality of affinity agents on the support structure, to contact the whole blood with the affinity agents; binding the target agent with the affinity agents; and removing the whole blood having a reduced amount of the target agent from the cartridge. The target agent is selected from the group consisting of: inhibitory checkpoint molecules, inflammatory factors, cancerous cells, autoantibodies, opioids and heavy metals.

### BACKGROUND

Hemodialysis is a process of purifying blood, typically used to treat patients whose kidneys are not functioning normally. This process removes waste products such as creatinine, urea and free water from the blood by pumping the patient's blood through a dialyzer to filter the blood. Dialyzers use cylindrical bundles of hollow fibers, whose walls are composed of semi-permeable membranes. Dialyzers are used in hemodialysis machines. The bundle of fibers is in a clear plastic cylindrical shell with four openings, or ports. One opening at each end of the cylinder communicates with each end of the bundle of hollow fibers. This forms the “blood compartment” of the dialyzer. Two other ports in the side of the cylinder communicate with the space around the hollow fibers, which is known as the “dialysate compartment.” Blood is pumped via the blood ports through this bundle of very thin capillary-like tubes, and the dialysate is pumped through the space surrounding the fibers causing the waste product to diffuse through the walls of the fibers into the dialysate. The waste products are removed using counter-current flow, where the blood flows in the opposite direction as the dialysate solution. The counter-current flow maintains the concentration gradient across the semi-permeable membrane, allowing for the diffusion of solutes across the semi-permeable membranes. Pressure gradients are applied when necessary to move fluid from the blood to the dialysate compartment.

FIG. 1 illustrates a diagram of a patient, blood pump, and a dialyzer, with the blood shown in the hollow fibers, surrounded by dialysate, and showing “dirty” blood entering the top port, and “clean” blood exiting the bottom port of the dialyzer, with the clean dialysate entering and dirty dialysate exiting to create the counter-current flow. FIG. 2 illustrates another dialyzer from FRESENIUS MEDICAL CARE® which shows the interior of the dialyzer compartment. FIG. 3 illustrates a dialyzer from FRESENIUS MEDICAL CARE®. FIG. 4 illustrates a dialyzer from B BRAUN® MEDICAL, INC. FIG. 5 illustrates the 2008T® Hemodialysis Machine, manufactured by FRESENIUS MEDICAL CARE®.

The pore size of the semi-permeable membranes determines the size of components that may be removed. Dialyzer membranes with smaller pore sizes are called “low-flux” and those with larger pore sizes are called “high-flux.” The goal of high-flux membranes is to pass relatively large molecules such as beta-2-microglobulin (MW 11,600 daltons), but not to pass albumin (MW ˜66,400 daltons). Every membrane has pores in a range of sizes. As pore size increases, some high-flux dialyzers begin to let key blood components pass out of the blood into the dialysate. Dialyzers are not able to selectively remove target agents.

Hemodialysis treatment and hemodialysis machines are extremely common in treatment facilities. Routine hemodialysis is often conducted in a dialysis outpatient facility or a purpose-built room in a hospital. Treatment is typically performed 3 to 4 days a week, lasting 3 to 6 hours per treatment. Modern hemodialysis machines are highly computerized and continuously monitor an array of safety-critical parameters, including blood and dialysate flow rates; dialysis solution conductivity, temperature, and pH; and analysis of the dialysate for evidence of blood leakage or presence of air. Dialysis is typically performed in a hospital setting, but dialysis machines for in-home use are also commercially available.

Removal of various undesirable components from whole blood is well known and often includes ex vivo separation of cellular components (for example, using centrifugation or filtration) to obtain a cell-free fluid that is then processed, typically using affinity media and/or enzymatic treatment. While such treatment is standard practice in many instances, hemolysis is often a problem.

Further known methods for binding of a contaminant from a fluid are described in WO 2011/005742 where a sorbent medium is modified and combined with the fluid. However, the sorbent medium must be separated from cellular components, which is in most cases not feasible to the required degree. To assist in separation of the sorbent, magnetic beads can be used as is described in U.S. Pat. No. 6,143,510. However, such methods are generally limited to ex vivo tests as residual quantities of magnetic beads are highly undesirable. To circumvent problems associated with removal of sorbent, selective permeable membranes may be employed as taught in US 2009/0114595. While such removal is conceptually simple, separation efficiency is in at least some cases less than desirable, especially where large molecules are separated.

Dialysis-like devices have been modified to include antibodies or proteins attached to hollow fiber membranes, in order to remove viruses, toxins or certain proteins from blood. To remove HIV virus and virus proteins, WO 2004/064608 describes immobilized lectin molecules within the porous exterior portion of the hollow fiber membranes of a dialyzer. WO 2015/095553 describes attaching antibodies to the inside of hollow fiber membranes to bind and remove uremic toxins. Devices without hollow fiber membranes have also been used to remove complement proteins from blood. WO 2010/030789 describes anti-complement antibodies and polymers covalently attached to a polymer matrix, in order to remove complement proteins. Another modified dialysis device is described in WO 2007/103572, which describes antibodies capable of binding exosomes of tumor cells to remove these exosomes from blood.

### SUMMARY

In a first aspect, the invention is a whole blood treatment device for treating a patient, including a cartridge configured to receive whole blood, having a wall defining an interior volume, an inlet, and an outlet, a support structure having a surface, in the cartridge, and an affinity agent attached to the surface of the support structure. The affinity agent is effective to bind a target agent, and the target agent is selected from the group consisting of: inhibitory checkpoint molecules, inflammatory factors, cancerous cells, autoantibodies, opioids and heavy metals.

In a second aspect, the invention is a method of removing a target agent from whole blood of a patient in a whole blood treatment device, including pumping whole blood into a cartridge, containing a support structure having a surface and a plurality of affinity agents on the support structure; contacting the whole blood with the affinity agents; binding the target agent with the affinity agents; removing the whole blood having a reduced amount of the target agent from the cartridge; and returning the whole blood having a reduced amount of the target agent to the patient. The affinity agent is effective to bind a target agent, and the target agent is selected from the group consisting of: inhibitory checkpoint molecules, inflammatory factors, cancerous cells, autoantibodies, opioids and heavy metals.

In a third aspect, the invention is a method of treating cancer, including pumping whole blood from a patient into a cartridge, containing a support structure having a surface and a plurality of affinity agents on the support structure, contacting the whole blood with the affinity agents, binding the target agent with the affinity agents, removing the whole blood having a reduced amount of the target agent from the cartridge, and returning the whole blood having a reduced amount of the target agent to the patient. The affinity agent is at least one inhibitory checkpoint molecule selected from the group consisting of: cytotoxic T-lymphocyte associated protein 4 (CTLA-4), programmed cell death-1 (PD-1), programmed death-ligand 1 (PD-L1), B7-1, B7-2, FOXP3+, FOXP3−, Treg 17, Tr1, Th3, IL-10 and TGF-β.

In a fourth aspect, the invention is a method of treating cancer, including pumping whole blood from a patient into a cartridge, containing a support structure having a surface and a plurality of affinity agents on the support structure, contacting the whole blood with the affinity agents, binding the target agent with the affinity agents, removing the whole blood having a reduced amount of the target agent from the cartridge, and returning the whole blood having a reduced amount of the target agent to the patient. The target agent is cancerous cells.

In a fifth aspect, the invention is a method of treating diseases associated with inflammation, including pumping whole blood from a patient into a cartridge, containing a support structure having a surface and a plurality of affinity agents on the support structure, contacting the whole blood with the affinity agents, binding the target agent with the affinity agents, removing the whole blood having a reduced amount of the target agent from the cartridge, and returning the whole blood having a reduced amount of the target agent to the patient. The target agent is at least one inflammatory factor selected from the group consisting of: IL-4, IL-10, TNFα, IL-17A, IL-17F, CRP, TNF, IL-1α, IL-1β, IL-5, IL-6, IL-8, IL-12, IL-23, CD2, CD3, CD20, CD22, CD52, CD80, CD86, C5 complement protein, BAFF, APRIL, IgE, α4β1 integrin and α4β7 integrin.

In a sixth aspect, the invention is a method of treating cancer, including administering chemotherapy to a patient having cancer, pumping whole blood from the patient into a cartridge, containing a support structure having a surface and a plurality of affinity agents on the support structure, contacting the whole blood with the affinity agents, binding the target agent with the affinity agents, removing the whole blood having a reduced amount of the target agent from the cartridge, and returning the whole blood having a reduced amount of the target agent to the patient. The target agent is at least one inhibitory checkpoint molecule selected from the group consisting of: cytotoxic T-lymphocyte associated protein 4 (CTLA-4), programmed cell death-1 (PD-1), programmed death-ligand 1 (PD-L1), B7-1, B7-2, FOXP3+, FOXP3−, Treg 17, Tr1, Th3, IL-10 and TGF-β.

In a seventh aspect, the invention is a method of regenerating a whole blood treatment device, including removing the blood from the whole blood treatment device, rinsing the whole blood treatment device with a regeneration fluid to unbind the target agents from the affinity agents, and sanitizing the whole blood treatment device.

### DEFINITIONS

The term “conjugated” means “chemically bonded to”.

The term “antibody” is used in the broadest sense as any antibody or protein including monoclonal antibodies, polyclonal antibodies, multi-specific antibodies, antibody fragments and chemically modified antibodies, where the chemical modification does not substantially interfere with the selectivity and specificity of the antibody or antibody fragment.

The term “aptamer” means oligonucleotide molecules that bind to a specific target agent.

The term “affinity agent” means an agent that has a binding affinity to the target agent to be removed.

The term “target agent” means any compound or agent that would be desirable to remove from blood.

The term “regeneration fluid” refers to a fluid used to remove the bound target agents from the affinity agent.

The term “hemodialysis system” refers to a machine or system that is capable of performing hemodialysis, hemofiltration and/or hemodiafiltration treatment to remove components from a patient's blood. A hemodialysis system may include pumps, sensors, water purification systems, and computer control systems. The term hemodialysis system does not include the dialyzer.

The term “dialyzer” means a filtration device having semi-permeable membranes used to remove excess wastes and fluid from the blood.

The term “anticoagulant” means any agent that disrupts blood coagulation.

### BRIEF DESCRIPTION OF THE DRAWINGS

FIG. 1 illustrates a diagram of a dialyzer in use for hemodialysis.

FIG. 2 illustrates a dialyzer from FRESENIUS MEDICAL CARE® which shows the interior of the dialyzer compartment.

FIG. 3 illustrates a dialyzer from FRESENIUS MEDICAL CARE®.

FIG. 4 illustrates a dialyzer from B BRAUN® MEDICAL, INC.

FIG. 5 illustrates the 2008T® Hemodialysis Machine manufactured by FRESENIUS MEDICAL CARE®.

FIG. 6 illustrates a whole blood treatment device.

FIG. 7 illustrates a whole blood treatment device with a portion cut-away to illustrate the beads inside the cartridge.

FIG. 8 illustrates a bead, with an affinity agent attached.

FIG. 9 illustrates a schematic of a patient, a hemodialysis system, and a whole blood treatment device.

FIG. 10 illustrates a diagram of the method of treating whole blood.

FIG. 11 illustrates a diagram of the method of regenerating a whole blood treatment device.

FIG. 12A illustrates the interior of a whole blood treatment device.

FIG. 12B illustrates the interior of a whole blood treatment device.

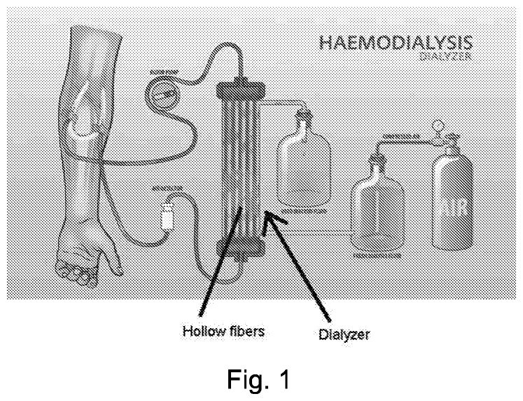

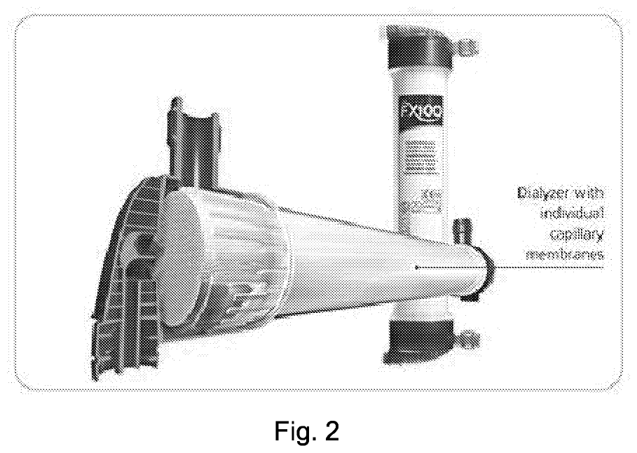

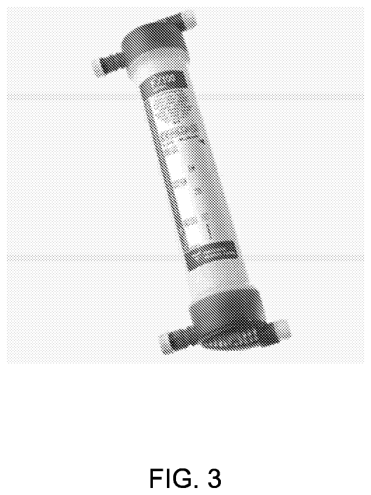

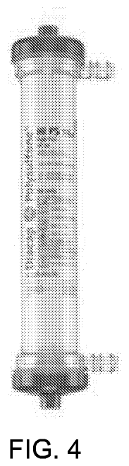

### DETAILED DESCRIPTION

The present application describes methods for selectively removing target agents from blood and a whole blood treatment device for selectively removing target agents from blood. The whole blood treatment device comprises a cartridge including a support structure having an affinity agent, which bind to a target agent in the blood, to remove the contaminant from circulation. The support structure may include beads, which provide a large surface area that allows for an amount of the affinity agent to be effective for the removal of a target agent from whole blood. Following the use to treat a patient, the support structure may be washed with a regeneration fluid to remove the bound target agent, allowing for reuse of the cartridge. The affinity agent may be selected to remove one or many different target agents in order to treat diseases or improve patient wellbeing. The whole blood treatment device is not limited by the size of a target agent, unlike dialyzer membranes, which are not able to selectively remove the desired components without potentially removing other important blood components as well.

The cartridge has a wall that defines an interior volume. Optionally, the wall may be coated with an anticoagulant. This anticoagulant avoids the buildup of clotting fibers inside the cartridge and the fouling of the support structure and cartridge. Alternatively, an anticoagulant may be added to the blood prior to the blood entering the whole blood treatment device. The amount of anticoagulant administered may be determined by sensors, and the amount of anticoagulant administered may be increased or decreased depending on the patient's needs. Whole blood, including the target agent, may be introduced into the cartridge. The target agent, having a much higher affinity to the affinity agent, will bind to the affinity agent, while the remaining blood will pass through the cartridge. The purified blood may be returned to the patient. The purified blood may also be stored for future use.

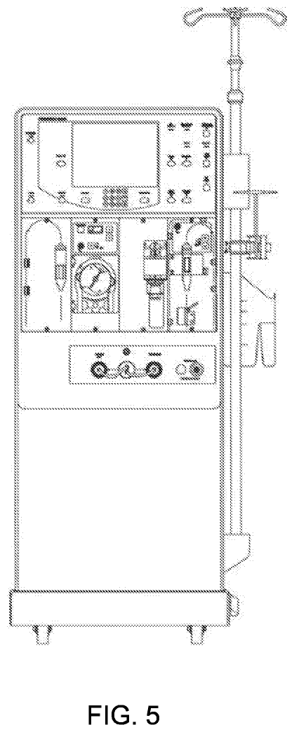

The whole blood treatment device may be configured to be received in a hemodialysis system, fitting into the normal position of a dialyzer. The use of the whole blood treatment device in existing hemodialysis systems avoids the need for hospitals and clinics to purchase additional blood filtering machinery, as typical hemodialysis systems include pumps, sensors, and other equipment that would be desirable for use with the whole blood treatment device. The hemodialysis system pumps blood from the patient into the whole blood treatment device. The whole blood treatment device removes selected target agents out of blood that is pumped through the device by the hemodialysis system, and the hemodialysis system would return the blood to the patient. Configuring the whole blood treatment device to be compatible with existing hemodialysis systems allows for reduced costs, and makes treatment using the whole blood treatment devices more convenient for patients. An example of a hemodialysis machine is the 2008T® Hemodialysis Machine, manufactured by FRESENIUS MEDICAL CARE®, as shown in FIG. 5. The inlet and outlet of the whole blood treatment device may have a quick-connectors, as described in 2008T® Hemodialysis Machine Operator's manual, for easy attachment and removal of the whole blood treatment device from the hemodialysis machine. Other means of attaching the cartridge to the hemodialysis machine would also be suitable, for example luer taper connections or tapered pipe threads.

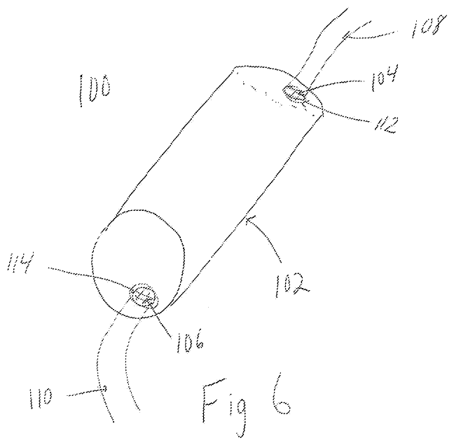

In FIG. 6, the whole blood treatment device, 100 is shown. This device includes a cartridge, 102, having a cylindrical shape. The cartridge includes an inlet 104 and an outlet, 106. The inlet, 104 has an inlet screen, 112 to prevent the beads from leaving the cartridge, 102. The outlet, 106 has an outlet screen, 114 to prevent the beads from leaving the cartridge, 102. The inlet is connected to an inlet tube, 108, bringing blood from the patient into the device, and the outlet is connected to an outlet tube, 110, returning blood to the patient after it has passed through the device and the desired target agents have been removed. The tubes are optionally connected to a hemodialysis system.

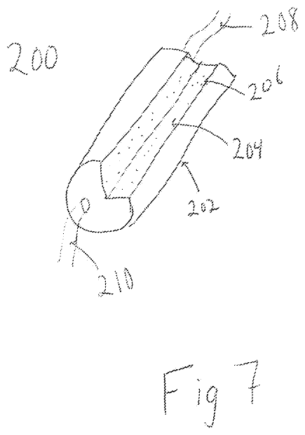

In FIG. 7, the whole blood treatment device, 200 is shown, with a cut away section removed to show the coated beads inside the cartridge. The outer wall of the cartridge, 202 is cut away to show the interior of the cartridge, 204. The beads, 206 are shown in the interior of the cartridge. An inlet tube, 208 is attached to the whole blood treatment device, 200 to bring blood into the device and an outlet tube, 210 is attached to the whole blood treatment device, 200 to provide an exit for blood that has been treated.

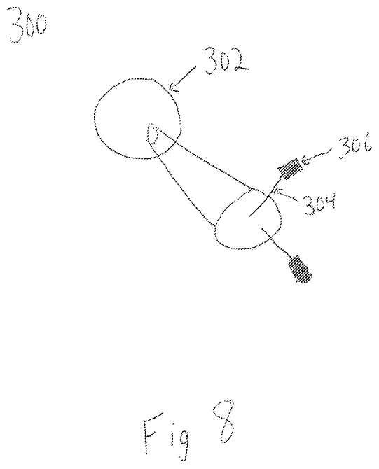

FIG. 8 illustrates the bead, 300 having a bead surface, 302. The affinity agent, 306 is attached to the bead surface via a linker, 304. The affinity agent binds the targeted agent, removing the target agent from the patient's blood.

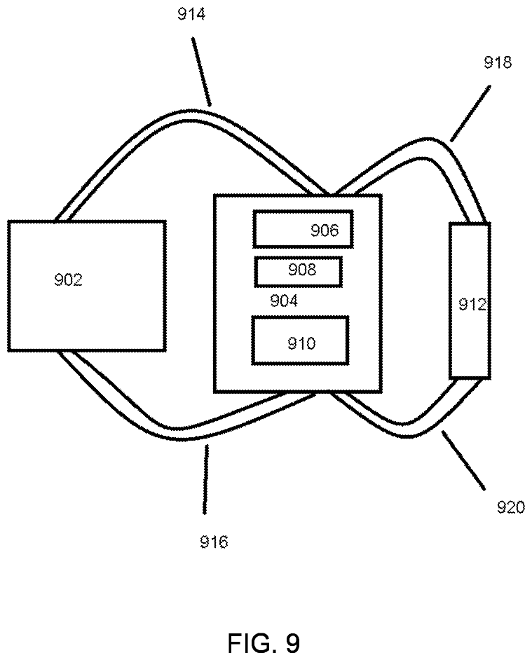

FIG. 9 illustrates schematic of a whole blood treatment device coupled to a hemodialysis system, and a patient. A patient, 902 is connected to a hemodialysis system, 904. The hemodialysis system includes at least one sensor, 906, a blood pump, 908 and a control circuit, 910 to control the hemodialysis system. The hemodialysis system is connected to a whole blood treatment device, 912. The patient is connected to the hemodialysis system by a patient blood withdrawal tube, 914. Blood that has been passed through the hemodialysis system and through the whole blood treatment device is returned to the patient through the patient blood return tube, 916. Blood is transported from the hemodialysis machine to the whole blood treatment device through the inlet tube, 918. After the blood has passed through the whole blood treatment device and the target agent has been removed, the blood returns to the hemodialysis system through the outlet tube, 920. The blood may also travel directly from the whole blood treatment device, 912 to the patient without passing through the hemodialysis system, 904.

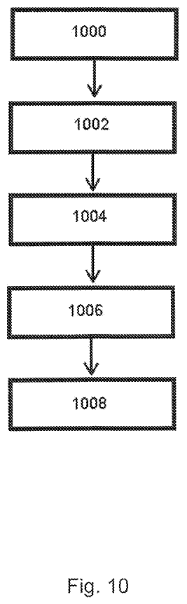

FIG. 10 illustrates a diagram of the method of treating whole blood. The first step comprises pumping the whole blood into a cartridge, 1000 containing a plurality of beads with a plurality of affinity agents on the beads. The next step comprises contacting the whole blood with the affinity agents, 1002. Following the contacting, the affinity agents selectively bind to the target agents, 1004. The last step comprises removing the blood from the cartridge, 1006. The blood may be returned to a patient or stored for later use, 1008.

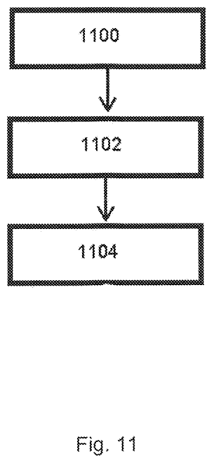

FIG. 11 illustrates a diagram of the method of regenerating a whole blood treatment device. The first step comprises removing the blood from the whole blood treatment device, 1100. The next step in the method comprises rinsing the device with a regeneration fluid to remove any attached target agents, 1102. For example, changing the pH, by allowing an acid or base to flow through the interior of the cartridge would cause the bound target agents to disassociate from the affinity agents. The last step comprises sterilizing the cartridge and the beads inside the cartridge to remove any contaminants and make the cartridge safe for reuse, 1104. Optionally, anticoagulant may be placed into the cartridge.

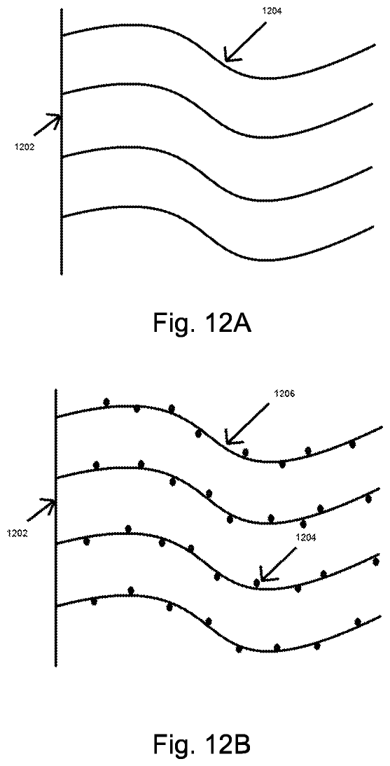

FIG. 12A illustrates the interior of a whole blood treatment device. The whole blood treatment device includes the interior wall of the cartridge, 1202. An irregularly shaped support structure, coated with affinity agent, 1204 is attached to the wall. FIG. 12B also illustrates the interior of a whole blood treatment device. Attached to the cartridge wall, 1202, is a fiber, 1206. Beads, 1204 are attached to the fiber along the length of the fiber.

Various target agents can be targeted for removal from blood, and various affinity agents can be used, where the affinity agents have a binding affinity to the target agent to be removed. The affinity agent may interact with the target agent with high specificity and selectivity through several different types of bonds and interaction. Such interactions include hydrogen bonding, ionic interaction, disulfide bridges, hydrophobic interaction, and other bonding types.

The device may be used to target various targets, such as proteins, fats, molecules, and ions, and may also be used to target cells, bacteria, viruses or parasites. The whole blood treatment device can also be used to treat various diseases that are caused by one or more target agents, or treat the symptoms of a disease caused by one or more of the target agents. Examples of these diseases include treating cancer by reducing inhibitory checkpoint molecules or removing cancer cells, treating autoimmune diseases by reducing inflammatory factors, cardiovascular disease by reducing low-density lipoprotein, treating metabolic diseases such as diabetes by reducing glucose, treating viral and bacterial infections by reducing the amount of virus, bacteria or associated toxin, and treating toxin exposure and heavy metal exposure by removing the toxin or heavy metal. Such diseases may be treated with the device of the present application by determining a target agent that can be removed from the body and determining an affinity agent that binds to the target agent. Preferably, antibodies or aptamers suited to binding the selected target agent could be used as the affinity agents, but it is understood that other compounds could also be used.

Methods of making antibodies are well-known in the art. Antibodies may be produced by immunizing animals and obtaining the antibodies from the animal serum. For example, polyclonal antibodies (pAbs) can be raised in a mammalian host by one or more injections of an immunogen, such as an extracellular domain of surface-expressed nucleolin, and, if desired, an adjuvant. Typically, the immunogen (and adjuvant) is injected in a mammal by a subcutaneous or intraperitoneal injection. The immunogen may include components such as polypeptides (isolated, non-isolated, or recombinantly produced), cells or cell fractions. Examples of adjuvants include Freund's complete and monophosphoryl Lipid A synthetic-trehalose dicorynomycolate (MPL-TDM). To improve the immune response, an immunogen may be conjugated to a polypeptide that is immunogenic in the host, such as keyhole limpet hemocyanin (KLH), serum albumin, bovine thyroglobulin or soybean trypsin inhibitor. Monoclonal antibodies (mAbs) may also be made by immunizing a host or lymphocytes from a host, harvesting the mAb-secreting (or potentially secreting) lymphocytes, fusing those lymphocytes to immortalized cells (for example, myeloma cells), and selecting those cells that secrete the desired mAb. Monoclonal antibodies are usually made by fusing mouse spleen cells immunized with a desired antigen with myeloma cells (B-cell cancer cells, which are known for producing antibodies). The fused cells are transferred to a medium that is selective for fused cells. Several cell cultures are then grown from single parent cells. The mAbs may be purified by conventional procedures such as protein A-sepharose, hydroxylapatite chromatography, gel electrophoresis, dialysis, ammonium sulfate precipitation or affinity chromatography. The antibodies may be whole antibodies and fragments or derivatives thereof. For example, when assaying live cells, using F_{ab} fragments will eliminate cross-linking, thus preventing the cells from endocytosing the bound antibodies.

Recombinant antibodies (rAbs) may also be used to bind to the target agent. rAbs are constructed in vitro using recombinant DNA technologies. The antibody genes may be isolated and then incorporated into plasmid DNA vectors, and the resulting plasmids are transferred into expression hosts such as bacteria, yeast, or mammalian cell lines. Transformation may be carried out chemically or by electroporation. Incorporated by reference are all the antibody production methods described in Frenzel et al., Expression of recombinant antibodies, Frontiers in Immunology, vol 4, article 20, 1-20 (2013).

It would be understood by those skilled in the art, that the systematic evolution of ligands by exponential enrichment (SELEX) technique, or similar techniques, could be used to produce aptamers that specifically bind or target agents or cells. In the SELEX technique, a large quantity of randomly generated sequences is generated and exposed to the target ligand. The sequences that did not bind the target ligand are removed through affinity chromatography. The sequences that did bind the target ligand are then eluted and amplified by PCR to prepare for subsequent rounds of selection, in order to find the sequence that binds best to the ligand. Incorporated by reference are all the techniques for making aptamers for various targets described in Lakhin, et al. “Aptamers: Problems, Solutions and Prospects”, Acta Naturae, vol. 5, pg 34-43 (2013).

The materials for the cartridge body include polypropylenes, polyethylenes, polycarbonates and polyamides. The cartridge body may be formed by any suitable manufacturing process, such as injection molding or extruding. The support structure may be fibers, beads or membranes. The support structure may be formed from agarose, cellulose, dextrin, polystyrene, polyethersulfone, polyvinyl difluoride, ethylene vinyl alcohol, polycarbonate, polyether, polyether carbonate, regenerated cellulose, cellulose acetate, polylactic acid, nylon, or polyurethane. Optionally, the cartridge includes hollow fiber membranes. The affinity agent may be attached to the inside of the hollow fiber or attached outside of the hollow fiber membranes. The cartridge may include hollow fiber membranes as described in WO 2004/064608.

Beads may be made from a variety of solid materials, including (1) metals and elements; (2) oxides; (3) semiconductors; and (4) polymers. Metals and elements, preferably non-magnetic metals and elements, include gold, silver, palladium, iridium, platinum and alloys thereof; elements include silicon, boron and carbon (such as diamond, graphene and carbon nanotubes), and solid compounds thereof. Oxides include titanium dioxide, silicon dioxide, zinc oxide, iron oxide, zirconium oxide, magnesium oxide, aluminum oxide and complex oxides thereof, such as barium titanate. Semiconductors include quantum dots, zinc sulfide, silicon/germanium alloys, boron nitride, aluminum nitride, and solid solutions thereof. Polymers include polyethylenes, polystyrenes, polyacrylamide, polyacrylates and polymethacrylates, and polysiloxanes. Preferably, the beads are non-toxic. The beads preferably have an average particle diameter of 1-1000 μm, preferably, 5-750 μm, more preferably 100-750 μm, including 150, 175, 200, 225, 250, 275, 300, 325, 350, 375, 400, 425, 450, 475, 500, 525, 550, 575, 600, 625, 650, 675, 700, 725 and 750 μm. The bead shapes may be spherical, oblong or various irregular shapes. The beads may have a diameter or smallest dimension of 1-1000 μm. For example, CELLTHRUBIGBEAD 300-500-micron beads, (Sterogene Bioseparations, Inc., Carlsbad, Calif., USA) allow blood passage through a packed column. Because shear forces can lyse cells, the proper size of the beads must be determined in order to avoid lysing cells. The flow rate of the blood through the device may be optimized to avoid cell lysing as well as consistent removal of the target agent.

The affinity agents may be conjugated to the support structure by well-known means. Oligonucleotides and proteins (including antibodies) have been attached to solid materials, such as metals and elements, oxides, semiconductors and polymers, by a variety of techniques. The chemical reactions that make attachment possible are well characterized and facilitate the attachment of biomolecules through their common chemical groups. The types of functionalities generally used for attachment include easily reactive components such as primary amines, sulfhydryls, aldehydes, and carboxylic acids (See Covalent Immobilization of Affinity Ligands, ThermoFisher Scientific, https://www.thermofisher.com/us/en/home/life-science/protein-biology/protein-biology-learning-center/protein-biology-resource-library/pierce-protein-methods/covalent-immobilization-affinity-ligands.html). The solid material is first activated with a compound that is reactive toward one or more of these functional groups. The activated material can then generate a covalent linkage between the affinity agent and material. These same techniques may be used to attached affinity agents to the support structure. When the affinity agent is an aptamer or an oligonucleotide it may have a 5′ prime thiol modification for attachment to a thiol linker, to connect the affinity agent to a support structure (see U.S. Pat. No. 9,452,219). The affinity agent may be attached to the support structure before the support structure is placed into the cartridge, or the entire cartridge may be prepared, including the support structure, and then the affinity agent is attached to the support structure. The affinity agent may be attached as described in WO 2004/064608.

One preferred affinity agent and bead composition is aptamers conjugated to gold particles or gold coated beads. Gold exhibits low toxicity, versatile surface chemistry, light absorbing/scattering properties, and tunable size. Aptamers effectively cap gold particles and prevent aggregation, are safe, stable, easy to synthesize, and non-immunogenic. Aptamers with 5′ prime thiol modification and or 3′ fluorophore Cy5 may be purchased. The thiol ends of aptamers may be reduced by tri(2-carboxyethyl) phosphine TECP (50 mM) which is active in slightly acidic pH 6.5 Tris-EDTA (10 mM) solution for 4-8 hours at room temperature. Gold nanoparticles may be purchased, for example from, NANOPARTZ and/or TED PELLA INC. Gold nanoparticles and aptamers may be mixed in the desired molar ratio at room temperature overnight for attachment. Excess reagents are then removed by centrifugation. In a similar fashion, gold coated beads, such as polymer beads coated with gold by sputtering may be attached to aptamers.

The number of affinity agents per bead may vary when the weight of the bead varies, even when the equivalent affinity agent concentration (or equivalent aptamer concentration) is otherwise the same. For example, the number of affinity agent molecules per bead may vary from 2 to 10,000, or 10 to 1000, including 20, 30, 40, 50, 60, 70, 80, 90, 100, 200, 300, 400, 500, 600, 700, 800 and 900.

The number of beads present in the cartridge may vary, and is preferably 10,000 to 1 million. More preferably a cartridge contains 50,000 to 200,000 beads. The beads may fill a volume of the cartridge from 10% to 90% of the volume, including 15%, 20%, 25%, 30%, 35%, 40%, 45%, 50%, 55%, 60%, 65%, 70%, 75%, 80%, and 85%.

The cartridge may have a volume of 20 to 500 ml, preferably having a volume of 50 to 100 ml, including 55, 60, 65, 70, 75, 80, 85, 90, and 95 ml. The cartridge preferably has dimensions that make it suitable for attachment to a hemodialysis machine.

In one preferred embodiment, the target agent to be removed is IL-8 and/or C-reactive protein (CRP). Chemotherapy compliance can be significantly improved when IL-8 and/or CRP concentrations are reduced in the blood of a patient that is treated with a drug that increases at least one of IL-8 and/or CRP.

In one particularly preferred aspect, IL-8 and CRP levels are elevated in whole blood of a patient undergoing taxane (for example, paclitaxel or decetaxel) chemotherapy, for example, in the treatment of pancreatic cancer. Such treatment may be in combination with other drugs, for example, gemzitabine. Most typically, chemotherapy in such and similar cases is not well tolerated and a significant fraction of patients will discontinue treatment due to the severe side effects.

IL-8 and CRP levels in patients about to discontinue chemotherapy is well above normal reference ranges, typically at least 25%, and most typically at least 50% above the upper range of normal, and continuous reduction of the elevated levels, preferably back to the reference range, will increase the level of compliance. The reduction of IL-8 and/or CRP will be carried out over a period that coincides with at least a portion of time over which elevated levels will be observed without treatment.

In this context, it should be noted that the reduction of IL-8 and/or CRP is not intended as a treatment modality of the underlying disease, but as a palliative treatment of a condition that is brought about by pharmacological intervention. Reduction of IL-8 and/or CRP improves subjective well-being of a patient, and especially relieves nausea, flu-like symptoms, loss of appetite, and physical and/or metal fatigue.

However, and with respect to the disease being treated, it should be appreciated that any disease that requires drug treatment, and/or any disease that is characterized by excessive blood concentrations of IL-8 and/or CRP is contemplated herein. For example, the methods may be effective not only in combating side effects of chemotherapy of various neoplastic diseases, but also infectious diseases and especially including viral diseases (and particularly influenza virus, H1N1 flu, SARS, etc.), chronic inflammatory diseases (e.g., COPD, rheumatoid arthritis, inflammatory bowel disease, psoriasis, etc.), atherosclerosis, and acute coronary syndrome.

Consequently, and depending on the particular nature of the disease or treatment, the chemotherapeutic agent may vary considerably. Most typically, however, the pharmaceutical treatment will include chemotherapy for various neoplastic diseases, and especially those agents that are known to be associated with an increase in IL-8 and/or CRP. For example, chemotherapies may include administration of one or more of receptor antibodies, alkylating agents, antimetabolites, microtubule inhibitors (and especially taxanes), topoisomerases, and kinase inhibitors. Various chemotherapy treatments are described in WO 2007/103572. Similarly, the methods may be implemented on an intermittent or continuous basis, and the reduction in IL-8 and/or CRP is performed as long as excessive levels of IL-8 and/or CRP are measured or anticipated. For example, where administration of a chemotherapeutic agent results in a relatively broad and temporary spike in blood IL-8 levels (for example, over 2 days), reduction of IL-8 may be performed in a continuous manner over two days. On the other hand, where the increase is relatively brief, reduction may be performed in a discontinuous manner immediately following administration of the chemotherapeutic agent. Reduction may be performed for at least three hours, more preferably at least 6 hours. Reduction may be performed for 1, 2, 3, 4, 5, 6, 7, 8, 9, 10 hours.

Removal is preferably monitored to achieve a continuous IL-8 and/or CRP level that is within the normal reference range (and in some cases below or slightly above). For example, the clinical range of IL-8 is typically between 10-80 pg/ml for a healthy person, and it is typically preferred that reduction of IL-8 is equal to or less than 100 pg/ml, and more preferably equal to or less than 80 pg/ml. Similarly, the clinical normal range for CRP is between 0-5 mg/l for male and 0-8 for female healthy adults, and it is typically preferred that reduction of CRP is equal to or less than 5 mg/l, more preferably equal to or less than 3 mg/l, and most preferably equal to or less than 1 mg/l.

Reduction of IL-8 and/or CRP is preferably affected via specific antibodies (for example, HuMab 10F8 for IL-8), or other known IL-8 binders, including glycosaminoglycan (GAG) heparin, protease inhibitor alpha2-macroglobulin, Cyclosporin A, however, engineered binding agents are also deemed suitable, including recombinant IL-8 receptor and fragments thereof. Similarly, it is contemplated that CRP can be reduced by binding of CRP to one or more types of monoclonal or polyclonal antibodies, Fc receptors (for example, FcγRIIa), etc.

Another target agent may be inhibitory checkpoint molecules. Inhibitory checkpoint molecules are secreted by cancer cells to reduce the immune response. There are several antibody drugs (referred to as immune checkpoint inhibitors or immune checkpoint blockades) that target these inhibitory checkpoint molecules in order to increase the immune response to cancer cells. Rather than using these antibodies as a drug, the whole blood treatment devices pass whole blood through the cartridge, containing a support structure with a surface modified with affinity agents (such as the antibody), where the inhibitory checkpoint molecules are bound by the affinity agents, and removed from the blood, reducing the concentration of inhibitory checkpoint molecules in the blood. Some examples of inhibitory checkpoint molecules are cytotoxic T-lymphocyte associated protein 4 (CTLA-4), programmed cell death-1 (PD-1), programmed death-ligand 1 (PD-L1), B7-1, B7-2, FOXP3+, FOXP3−, Treg 17, Tr1, Th3, IL-10, and TGF-β. Some examples of antibodies that could be used as affinity agents include ipilimumab, ticilimumab, pembrolizumab, atezolizumab and nivolumab, along with many others, as described in Ghirelli et al. (“Targeting immunosuppression for cancer therapy” J Clin Invest. 2013; 123 (6):2355-2357.). Incorporated by reference are all the antibodies for use as immune checkpoint blockade or immune checkpoint inhibitors listed in Darvin et al., “Immune checkpoint inhibitors: recent progress and potential biomarkers” Experimental & Molecular Medicine vol. 50, Article number: 165 (2018). Also incorporated by reference are all the antibodies described in Ghirelli et al. (“Targeting immunosuppression for cancer therapy” J Clin Invest. 2013; 123 (6):2355-2357.). Incorporated by reference are all the molecules for use as immune checkpoint blockade or immune checkpoint inhibitors listed in González-Rodríguez et al., Immune checkpoint inhibitors: review and management of endocrine adverse effects, Oncologist vol 21 p. 804-816 (2016). The patients receiving treatment to remove inhibitory checkpoint molecules may be receiving chemotherapy treatment or have previously received chemotherapy treatment, or may subsequently receive chemotherapy treatment.

Inflammatory factors are often reduced using antibody drug treatments. Rather than administering antibodies as drugs, these same antibodies can be used in the cartridge of the whole blood treatment devices to bind inflammatory factors to reduce the concentration of inflammatory factors in the blood. Inflammatory factors may include IL-4, IL-10, TNFα, IL-17A, IL-17F, CRP, TNF, IL-1α, IL-16, IL-5, IL-6, IL-8, IL-12, IL-23, CD2, CD3, CD20, CD22, CD52, CD80, CD86, C5 complement protein, BAFF, APRIL, IgE, α4β1 integrin and α4β7 integrin. These inflammatory factors may be bound by IL-17A/F antibodies, abatacept, alefacept, alemtuzumab, atacicept, belimumab, canakinumab, eculizumab, epratuzumab, natalizumab, ocrelizumab, ofatumumab, omalizumab, otelixizumab, rituximab, teplizumab, vedolizumab, adalimumab, briakinumab, certolizumab pegol, etanercept, golimumab, infliximab, mepolizumab, reslizumab, tocilizumab and ustekinumab. Other antibodies or aptamers could also be used to target inflammatory factors. Incorporated by reference are the antibodies described in Focosi et al. (“Immunosuppressive monoclonal antibodies: current and next generation” Clin Microbiol Infect 2011; 17: 1759-1768) and Chan, A. C. et al. (“Therapeutic antibodies for autoimmunity and inflammation”, Nature Reviews Immunology, vol. 10, pp. 301-316, (2010)). Diseases that could be treated by targeting inflammatory factors include asthma, rheumatoid arthritis, autoimmune disorders and gastrointestinal diseases. Sepsis may be treated by the removal of various potent cytokines, including tumor necrosis factor (TNF) and interleukin 1, as well as inhibitory checkpoint molecules.

Autoantibodies can be targeted for removal in order to treat the symptoms of autoimmune disorders. Autoantibodies play a pivotal role in the pathogenesis of many diseases and autoantibodies mediate both systemic inflammation and tissue injury. Diseases that could be treated by removal of autoantibodies include Rheumatoid arthritis, Systemic lupus erythematosus (lupus), Inflammatory bowel disease (IBD), Multiple sclerosis (MS), Type 1 diabetes mellitus, Guillain-Barre syndrome, Graves' disease, and Psoriasis. Incorporated by reference are all the antigens described in Suurmond et al., “Autoantibodies in systemic autoimmune diseases: specificity and pathogenicity” Journal of clinical investigation vol. 125, 6 (2015): 2194-202. Also incorporated by reference are all the antigens described in Rowley et al. “The Role of Pathogenic Autoantibodies in Autoimmunity” Antibodies, vol. 4, pg. 314-353 (2015).

Viruses can be targeted using various affinity agents. Removing viruses from the blood would avoid the need to administer various drugs to a patient, which would avoid unwanted side effects from these drugs. The target agents can include viruses and cells infected with viruses. Examples of viruses include chickenpox, flu (influenza), herpes, human immunodeficiency virus (HIV/AIDS), human papillomavirus (HPV), infectious mononucleosis, mumps, measles, rubella and shingles. Antibodies that bind to these viruses may be used as the affinity agent and can be prepared by well-known methods.

Antibodies can be used to treat various cancers. Rather than using antibodies that bind cancer cells as a drug, these antibodies can be coated onto the support structure of the whole blood treatment devices. Examples of cancer cells include skin, breast, lung, pancreas, kidney, leukemia, lymphoma, and many other types of cancer. For example, treatment of cancer can be carried out by targeting cells displaying nucleolin on the cell surface. Anti-nucleolin antibodies can bind to the nucleolin on the surface of the cancer cell. Various anti-nucleolin antibodies include p7-1A4 mouse monoclonal antibody, sc-8031 mouse mAb, sc-9893 goat polyclonal Ab (pAb), sc-9892 goat pAb, clone 4E2 mouse mAb, and clone 3G4B2 mouse mAb, which may be used as affinity agents. AS1411, an oligonucleotide that binds nucleolin, may also be used. Other examples of antibody drugs to treat cancer cells, which may be used as affinity agents include alemtuzumab, trastuzumab, Ibritumomab tiuxetan, brentuximab vedotin, ado-trastuzumab emtansine, denileukin diftitox, and blinatumomab, along with others (and hereby incorporated by reference) can be found in American Cancer Society; Monoclonal Antibodies to Treat Cancer (available at www.cancer.org/treatmentltreatments-and-side-effects/treatment-types/immunotherapy/monoclonal-antibodies.html).

Heavy metal poisoning may be treated with a whole blood treatment device to reduce the concentration of heavy metals to a safe level. Heavy metals may include lead, mercury, arsenic and cadmium. These metals can be bound by an affinity agent such as DNAzyme (hereby incorporated by reference) as described in Zhang et al. (“Metal Ion Sensors Based on DNAzymes and Related DNA Molecules” Annual review of analytical chemistry 2011; 4 (1):105-128). Incorporated by reference are also the aptamers for binding metal ions described in Liu, et al. “Rational Design of “Turn-On” allosteric DNAzyme Catalytic Beacons for Aqueous Mercury Ions with Ultrahigh Sensitivity and Selectivity” Angew. Chem. Int. Ed. 2007, 46, 7587-7590, and Li, et al., “A highly Sensitive and Selective Catalyst DNA Biosensor for Lead Ions” J. Am. Chem. Soc., 2000, 122 (42), pp 10466-10467. Also incorporated by reference are the aptamers described in Qu et al. “Rapid and Label-Free Strategy to Isolate Aptamers for Metal Ions” ACS nano vol. 10 (8), pg. 7558-65 (2016). Chelating agents could also be used as an affinity agent to bind to the target agents in the device.

Alzheimer's disease can be treated with solanezumab. Rather than administering this drug to a patient, a whole blood treatment device can be used to filter target agents from whole blood, using the antibody as an affinity agent. Some examples of target agents for Alzheimer's disease include misfolded amyloid beta and tau proteins.

Bacteria and toxins in the bloodstream can be treated with various drugs. Rather than administering these drugs to a patient, a whole blood treatment device can be used to filter the bacteria or toxins from whole blood. Examples of toxins which could be target agents include botulinum toxins produced by Clostridium botulinum, Clostridium difficile toxins produced by Clostridium difficile, corynebacterium diphtheriae toxins produced during life-threatening symptoms of diphtheria, and tetanospasmin produced by Clostridium tetani. Antibodies and aptamers may be used as affinity agents for the bacteria or toxins. Such antibodies and aptamers may be prepared by well-known methods. Incorporated by reference are all the antibodies that bind to toxins in U.S. Pat. Nos. 10,160,797 and 10,117,933.

Methanol poisoning can be treated by competitive inhibition. Methanol concentration could also be reduced using the whole blood treatment device with affinity agents such as aptamers specific for methanol.

Opioid overdoses can be treated by administering antibodies that bind to the opioid molecules and prevent the opioid from attaching to opioid receptors. Rather than administering these drugs, a whole blood treatment device could be used to reduce the concentration of opioids in the blood. Antibodies, aptamers or other compounds may be used as affinity agents for heroin, fentanyl, or methamphetamines. Incorporated by reference are the antibodies for binding methamphetamines described in Owens et al., “Monoclonal antibodies as pharmacokinetic antagonists for the treatment of (+)-methamphetamine addiction”, CNS Neurol Disord Drug Targets. 2011; 10 (8):892-8. Incorporated by reference are the antibodies described in Banks et al., “Immunopharmacotherapies for treating opioid use disorder” Cell Science & Society Series: Opioid Crisis, Volume 39, ISSUE 11, pg. 908-911 (2018).

The regeneration fluid may be administered to the whole blood treatment device, following the use of the device to remove contaminants from whole blood. The regeneration fluid may be administered for a length of time to sufficiently clear the target agents. Other cleaners and disinfectants may also be used to clean the device between uses. The regeneration fluid may be any fluid that is capable of elution of the target agent. This fluid may be a low pH buffer, a high salt concentration, or a high concentration of a competitive agent that binds affinity agent, thus allowing the target agents to be released. The pH can be adjusted with acids or bases, such as hydrochloric acid or sodium hydroxide. In one embodiment the support structure is beads. The beads may be replaced by removing the beads from the cartridge, sterilizing the cartridge, and providing new beads.

The anticoagulant may be heparin. The heparin may be unfractionated heparin or low-molecular-weight heparin preparations. Anticoagulant alternatives to heparin include danaparoid, lepirudin, and argatroban. Citrate anticoagulation may also be used in the whole blood treatment device to prevent coagulation. The anticoagulant may coat the walls of the cartridge. Alternatively, an anticoagulant may be added to the blood prior to the blood entering the whole blood treatment device. The amount of anticoagulant administered may be determined by sensors, and the amount of anticoagulant administered may be increased or decreased depending on the patient's needs.

### REFERENCES

1. WO 2011/005742

2. U.S. Pat. No. 6,143,510

3. US 2009/0114595

4. Ghirelli et al. (“Targeting immunosuppression for cancer therapy” J Clin Invest. 2013; 123 (6):2355-2357.)

5. Focosi et al. (“Immunosuppressive monoclonal antibodies: current and next generation” Clin Microbiol Infect 2011; 17: 1759-1768)

6. Chan, A. C. et al. (“Therapeutic antibodies for autoimmunity and inflammation”, Nature Reviews Immunology, vol. 10, pp. 301-316, (2010))

7. American Cancer Society; Monoclonal Antibodies to Treat Cancer (available at www.cancer.org/treatmentltreatments-and-side-effects/treatment-types/immunotherapy/monoclonal-antibodies.html)

8. Hotchkiss et al. (“Immunosuppression in sepsis: a novel understanding of the disorder and a new therapeutic approach” The Lancet infectious diseases. 2013; 13 (3):260-268)

9. “Affinity Chromatography” Wikipedia (https://en.wikipedia.org/wiki/Affinity_chromatography) viewed on May 7, 2018.

10. U.S. Pat. No. 4,500,309

11. Fischer, K. G., “Essentials of anticoagulation”, Hemodialysis international, vol. 11, issue 2 pp. 178-189 (2007)

12. Frenzel et al., Expression of recombinant antibodies. Frontiers in Immunology, vol 4, article 20, 1-20 (2013).

13. Darvin et al., “Immune checkpoint inhibitors: recent progress and potential biomarkers” Experimental & Molecular Medicine vol. 50, Article number: 165 (2018).

14. González-Rodríguez et al., Immune checkpoint inhibitors: review and management of endocrine adverse effects. Oncologist vol 21 804-816 (2016).

15. 2008T Hemodialysis Machine Operator's manual, Fresenius Medical Care (2008)

16. Lakhin, et al. “Aptamers: Problems, Solutions and Prospects”, Acta Naturae, vol. 5, pg 34-43 (2013).

17. Owens et al., Monoclonal antibodies as pharmacokinetic antagonists for the treatment of (+)-methamphetamine addiction, CNS Neurol Disord Drug Targets. 2011; 10 (8):892-8.

18. Lakhin, et al. “Aptamers: Problems, Solutions and Prospects”, Acta Naturae, vol. 5, pg 34-43 (2013).

19. Covalent Immobilization of Affinity Ligands, ThermoFisher Scientific, https://wWw.thermofishercom/us/en/home/life-science/protein-biology/protein-biology-learning-center/protein-biology-resource-library/pierce-protein-methods/covalent-immobilization-affinity-ligands.html).

20. U.S. Pat. No. 9,452,219

21. Suurmond et al., “Autoantibodies in systemic autoimmune diseases: specificity and pathogenicity” Journal of clinical investigation vol. 125, 6 (2015): 2194-202.

22. List of autoimmune diseases” Wikipedia, https://en.wikipedia.org/wiki/List_of_autoimmune_diseases visited on Jan. 24, 2019.

23. Zhang et al. (“Metal Ion Sensors Based on DNAzymes and Related DNA Molecules” Annual review of analytical chemistry 2011; 4 (1):105-128).

24. Liu, et al. “Rational Design of “Turn-On” allosteric DNAzyme Catalytic Beacons for Aqueous Mercury Ions with Ultrahigh Sensitivity and Selectivity” Angew. Chem. Int. Ed. 2007, 46, 7587-7590.

25. Li, et al., “A highly Sensitive and Selective Catalyst DNA Biosensor for Lead Ions” J. Am. Chem. Soc., 2000, 122 (42), pp 10466-10467.

26. Qu et al. “Rapid and Label-Free Strategy to Isolate Aptamers for Metal Ions” ACS nano vol. 10 (8), pg. 7558-65 (2016).

27. U.S. Pat. No. 8,709,428

28. Owens et al., “Monoclonal antibodies as pharmacokinetic antagonists for the treatment of (+)-methamphetamine addiction”, CNS Neurol Disord Drug Targets. 2011; 10 (8):892-8.

29. Banks et al., “Immunopharmacotherapies for treating opioid use disorder” Cell Science & Society Series: Opioid Crisis, Volume 39, ISSUE 11, pg. 908-911 (2018).

30. Rowley et al. “The Role of Pathogenic Autoantibodies in Autoimmunity” Antibodies, vol. 4, pg. 314-353 (2015).

31. U.S. Pat. No. 10,160,797.

32. U.S. Pat. No. 10,117,933.

33. Tullis et al., “Affinity Hemodialysis for Antiviral Therapy. I. Removal of HIV-1 from Cell Culture Supernatants, Plasma, and Blood”, Therapeutic apheresis and dialysis, vol. 6, no. 3, pages 213-220 (2002).

34. Tullis et al., “Affinity hemodialysis for antiviral therapy. II. Removal of HIV-1 viral proteins from cell culture supernatants and whole blood”, Blood purification, KARGER, BASEL, vol. 21, no. 1, pages 58-63 (2003).

35. WO 2004/064608.

36. WO 2007/103572.

37. WO 2015/095553.

38. US 2019/117869.

39. WO 2010/030789.

40. Marleau et al., “Exosome removal as a therapeutic adjuvant in cancer”, Journal of Translational Medicine, BioMed Central, vol. 10, no. 1, (2012).

1. A whole blood treatment device for treating a patient, comprising:
a cartridge configured to receive whole blood, having a wall defining an interior volume, an inlet, and an outlet,
a support structure having a surface, in the cartridge, and
an affinity agent attached to the surface of the support structure,
wherein the affinity agent is effective to bind a target agent, and
the target agent is selected from the group consisting of: inhibitory checkpoint molecules, inflammatory factors, cancerous cells, autoantibodies, opioids and heavy metals.

2. The whole blood treatment device of claim 1, wherein the target agent is at least one inhibitory checkpoint molecule selected from the group consisting of: cytotoxic T-lymphocyte associated protein 4 (CTLA-4), programmed cell death-1 (PD-1), programmed death-ligand 1 (PD-L1), B7-1, B7-2, FOXP3+, FOXP3−, Treg 17, Tr1, Th3, IL-10 and TGF-β.

3. The whole blood treatment device of claim 1, wherein the target agent is at least one inflammatory factor selected from the group consisting of: IL-4, IL-10, TNFα, IL-17A, IL-17F, CRP, TNF, IL-1αIL-1β, IL-5, IL-6, IL-8, IL-12, IL-23, CD2, CD3, CD20, CD22, CD52, CD80, CD86, C5 complement protein, BAFF, APRIL, IgE, α4β1 integrin and α4β7 integrin.

4. The whole blood treatment device of claim 1, wherein the target agent is selected from the group consisting of IL-8, CRP and mixtures thereof.

5. The whole blood treatment device of claim 1, wherein the target agent is cancerous cells.

6. The whole blood treatment device of claim 1, wherein the support structure comprises a plurality of beads.

7. The whole blood treatment of claim 6, further comprising an inlet screen over the inlet, and
an outlet screen over the outlet.

8. The whole blood treatment device of claim 1, wherein the device is configured to couple to a hemodialysis system.

9. The whole blood treatment device of claim 8, wherein the hemodialysis system comprises:
a pump,
a sensor, and
an inlet tube, connecting the hemodialysis system to the whole blood treatment device,
an outlet tube, connecting the whole blood treatment device to the hemodialysis system,
a patient blood withdrawal tube, connecting a patient to the hemodialysis system, and
a patient blood return tube, connecting the hemodialysis system to the patient.

10. (canceled)

11. (canceled)

12. The whole blood treatment device of claim 1, wherein the affinity agent is an aptamer.

13. The whole blood treatment device of claim 1, wherein the affinity agent is an antibody.

14. The whole blood treatment device of claim 2, wherein the target agent is selected from the group consisting of: PD-L1, PD-1, CTLA-4 and mixtures thereof.

15. The whole blood treatment device of claim 2, wherein the affinity agent is selected from the group consisting of: ipilimumab, ticilimumab, pembrolizumab, atezolizumab, nivolumab and mixtures thereof.

16. The whole blood treatment device of claim 1, further comprising an anticoagulant in the cartridge.

17. The whole blood treatment device of claim 6, wherein the beads comprise gold, the affinity agent is an aptamer, and the whole blood treatment device is configured to couple to a hemodialysis system.

18. A method of removing a target agent from whole blood of a patient in a whole blood treatment device, comprising:
pumping whole blood into a cartridge, containing a support structure having a surface and a plurality of affinity agents on the support structure, contacting the whole blood with the affinity agents,
binding the target agent with the affinity agents,
removing the whole blood having a reduced amount of the target agent from the cartridge, and
returning the whole blood having a reduced amount of the target agent to the patient,
wherein the target agent is selected from the group consisting of: inhibitory checkpoint molecules, inflammatory factors, cancerous cells, autoantibodies, opioids and heavy metals.

19. A method of treating cancer, comprising:
pumping whole blood from a patient into a cartridge, containing a support structure having a surface and a plurality of affinity agents on the support structure, contacting the whole blood with the affinity agents,
binding the target agent with the affinity agents,
removing the whole blood having a reduced amount of the target agent from the cartridge, and
returning the whole blood having a reduced amount of the target agent to the patient,
wherein the target agent is at least one inhibitory checkpoint molecule selected from the group consisting of: cytotoxic T-lymphocyte associated protein 4 (CTLA-4), programmed cell death-1 (PD-1), programmed death-ligand 1 (PD-L1), B7-1, B7-2, FOXP3+, FOXP3−, Treg 17, Tr1, Th3, IL-10 and TGF-β.

20. A method of treating cancer, comprising:
pumping whole blood from a patient into a cartridge, containing a support structure having a surface and a plurality of affinity agents on the support structure, contacting the whole blood with the affinity agents,
binding the target agent with the affinity agents,
removing the whole blood having a reduced amount of the target agent from the cartridge, and
returning the whole blood having a reduced amount of the target agent to the patient,
wherein the target agent is cancerous cells.

21. A method of treating diseases associated with inflammation, comprising:
pumping whole blood from a patient into a cartridge, containing a support structure having a surface and a plurality of affinity agents on the support structure, contacting the whole blood with the affinity agents,
binding the target agent with the affinity agents,
removing the whole blood having a reduced amount of the target agent from the cartridge, and
returning the whole blood having a reduced amount of the target agent to the patient,
wherein the target agent is at least one inflammatory factor selected from the group consisting of: IL-4, IL-10, TNFα, IL-17A, IL-17F, CRP, TNF, IL-1α, IL-1β, IL-5, IL-6, IL-8, IL-12, IL-23, CD2, CD3, CD20, CD22, CD52, CD80, CD86, C5 complement protein, BAFF, APRIL, IgE, α4β1 integrin and α4β7 integrin.

22. (canceled)

23. The method of claim 19, wherein the affinity agent is selected from the group consisting of: ipilimumab, ticilimumab, pembrolizumab, atezolizumab, nivolumab and mixtures thereof.

24. (canceled)

25. The method of claims 21, wherein the affinity agent is selected from the group consisting of: IL-17A/F antibodies, abatacept, alefacept, alemtuzumab, atacicept, belimumab, canakinumab, eculizumab, epratuzumab, natalizumab, ocrelizumab, ofatumumab, omalizumab, otelixizumab, rituximab, teplizumab, vedolizumab, adalimumab, briakinumab, certolizumab pegol, etanercept, golimumab, infliximab, mepolizumab, reslizumab, tocilizumab, ustekinumab and mixtures thereof.

26. A method of making the whole blood treatment device of claim 1, comprising:
coating a support structure with an affinity agent, and
placing the support structure inside a cartridge, wherein the cartridge has an inlet and an outlet.

27-29. (canceled)

30. the method of claim 18, further comprising regenerating the whole blood treatment device, wherein the method of regenerating comprises:
removing the blood from the whole blood treatment device,
rinsing the whole blood treatment device with a regeneration fluid to unbind the target agents from the affinity agents, and
sterilizing the whole blood treatment device.

31. The method of claim 19, wherein chemotherapy treatment has been administered to the patient.
